(19) United States
(12) Patent Application Publication
(10) Pub. No.: US 2007/0093700 A1
(43) Pub. Date: Apr. 26, 2007

(54) Computational optical biopsy

(76) Inventors: Ge Wang, Iowa City, IA (US); Yi Li, Iowa City, IA (US); Ming Jiang, Iowa City, IA (US); Wenxiang Cong, Iowa City, IA (US); Xin Quian, Iowa City, IA (US)

Correspondence Address: NEEDLE & ROSENBERG, P.C., SUITE 1000, 999 PEACHTREE STREET, ATLANTA, GA, 30309-3915, US

(21) Appl. No.: 11/444,282

(22) Filed: May 31, 2006

Related U.S. Application Data
(60) Provisional application No. 60/685,783, filed on May 31, 2005.

(51) Int. Cl. A61B 5/00

(52) U.S. Cl. 600/317; 600/309; 600/314

(57) ABSTRACT

Methods, systems and apparatuses for reconstructing a light source distribution or estimating a light source feature within a subject include an optical data receiving mechanism that is positionable at least at one location within the subject and is configured to acquire signal data from a light source located within a subject. A computational device is configured to receive signal data acquired by the optical data receiving mechanism and to reconstruct the light source distribution or to estimate a light source feature from at least a portion of the received data.

### CROSS-REFERENCE TO RELATED APPLICATIONS

This application claims priority to U.S. Provisional Application Number 60/685,783, filed on May 31, 2005. The aforementioned application is herein incorporated by reference in its entirety.

### ACKNOWLEDGEMENTS

This invention was made with government support under Grant EB001685 awarded by the National Institutes of Health/National Institute for Biomedical Imaging and Bioengineering. The government has certain rights in the invention.

### BACKGROUND OF THE INVENTION

Gene therapy is a breakthrough in modern medicine, which promises to cure diseases by modifying gene expression. Major efforts are being made to understand the linkage of genes to phenotypic expression for the development of molecular medicine. An important component of this perspective is small animal imaging that allows in vivo studies at anatomical, functional, cellular and molecular levels.

A key for development of gene therapy is to monitor the in vivo gene transfer and its efficacy in a small animal model. To map the distribution of an administered gene, reporter genes such as those producing luciferase are being used to generate light signals within living mice, which can be externally measured. Optical imaging of small animals based on fluorescent/bioluminescent probes promises great opportunities for translational research and eventually clinical applications because fluorescent/bioluminescent signals directly reveal molecular and cellular activities, and are sensitive, specific, non-ionizing, non-invasive and cost-effective.

Little research has been done for optical molecular imaging of human patients. Light sources induced by fluorescence or bioluminescence probes are usually weak, and would be deep inside a body if used in patients. Optical methods for in vivo imaging are all faced with the problem of limited transmission of light through tissues. Because the human body absorbs and scatters photons in the visible and near infrared ranges with the mean-free-path in the sub-millimeter domain, such a source cannot be effectively estimated/reconstructed based on optical flux measures on the body surface.

### SUMMARY OF THE INVENTION

Methods, systems and apparatuses for reconstructing a light source distribution or estimating a light source feature within a subject are provided herein.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate aspects of the invention and together with the description, serve to explain the principles of the invention.

FIG. 1A is a schematic diagram illustrating an exemplary optical data receiving mechanism and exemplary components of an exemplary computational optical biopsy system.

FIG. 1B is a schematic diagram illustrating an exemplary optical data receiving mechanism.

FIG. 2 is a block diagram illustrating an exemplary computational optical biopsy system based on fluorescence.

FIG. 3 is a block diagram illustrating an exemplary computational optical biopsy system based on bioluminescence.

FIG. 4 is a flow chart illustrating an exemplary method of computing light source/tissue parameters.

FIG. 5 a-d show exemplary data produced using the described systems and methods.

### DETAILED DESCRIPTION OF THE INVENTION

The present invention may be understood more readily by reference to the following detailed description and the Examples included herein and to the Figures and their previous and following descriptions.

Before the present apparatuses, systems, and methods are disclosed and described, it is to be understood that this invention is not limited to specific synthetic methods, specific algorithms, or to particular system components, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a light source” includes mixtures of light sources; reference to “a computational device” includes embodiments comprising two or more such devices, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of the ranges are significant both in relation to the other endpoints, and independently of the other endpoints.

“Optional” or “optionally” means that the subsequently described event or circumstance may or may not occur, and that the description includes instances where said event or circumstance occurs and instances where it does not. For example, the phrase “optionally a laser light source is used” means that the laser light source may or may not be used and that the description includes systems wherein a laser light source is used and wherein a laser light source is not used.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In one aspect, the subject is a mammal such as a primate or a human.

Provided herein are systems, apparatuses and methods for estimating a feature or parameter of a light source and for determining or reconstructing the light source distribution of a light source located within a subject using computation.

In contrast to current optical imaging techniques, the systems, apparatuses and methods disclosed herein can utilize optics and computation to sense, estimate, determine and/or reconstruct an underlying source intensity distribution or extract, or estimate its features of interest such as, but not limited to, source center, total energy, moment features, and statistical indexes.

An apparatus for estimating and/or reconstructing a light source feature and/or distribution within a subject can comprise an optical data receiving mechanism. The optical data receiving mechanism can be positionable at least at one location within the subject and can be configured to acquire signal data from a light source positioned within the subject. In one aspect, the optical data receiving mechanism can be operatively connected to a computational device for transmitting at least a portion of the acquired signal data to a computational device. In a further aspect, the computational device can be configured to estimate and/or reconstruct a feature and/or distribution of the light source from at least a portion of the transmitted data.

An exemplary method can comprise positioning an optical data receiving mechanism within the subject. In this aspect, the optical data receiving mechanism can acquire signal data from the light source. Further, at least a portion of the acquired signal data can be transmitted from the optical data receiving mechanism to a computational device and the distribution and/or feature of the light source can be determined or estimated from at least a portion of the transmitted data by estimating and/or reconstructing the feature and/or distribution of the light source.

Another exemplary method can comprise positioning an optical data receiving mechanism within the subject. In this aspect, the optical data receiving mechanism can acquire signal data from the light source. In a further aspect, anatomical data can be acquired from the subject and positional data is acquired from the optical data receiving mechanism located within the subject. At least a portion of the acquired signal data, anatomical data, and positional data can be transmitted to a computational device. In another aspect, the distribution and/or feature of the light source can be determined or estimated from at least a portion of the transmitted data by estimating and/or reconstructing the distribution of the light source.

In various aspect, the systems, apparatus and methods can be used for functional, cellular and molecular imaging of a subject. In practice, an optical data receiving mechanism, which may be referred to herein as a “biopsy needle,” “optical biopsy needle,” “needle,” or “optical fiber biopsy probe” can be inserted into the subject's tissue towards and/or through a “region of interest,” “desired target,” or “target region.” As used herein, a region of interest refers to a portion of a subject's body comprising a light source and its neighborhood, which is any portion of a subject's body that is in proximity to a region comprising a light source such that light signal data can be collected, or acquired as described herein. Signal modeling and estimation algorithms can be applied to data collected along at least one biopsy needle trajectory. Target source intensity distributions or features produced by a light source or sources located within a subject can be triggered by molecular probes instead of tissue/vascular properties. Both fluorescent and bioluminescent sources can be used. Additional optical probes that are functionally similar to that of fluorescence and/or bioluminescence can also be used.

Thus, the disclosed systems, apparatuses and methods can be used to detect a bioluminescent or fluorescent light source inside a large volume, such as a human's body. Such a light source can be used to label cellular and molecular targets within the subject including, but not limited to, features of cells, markers of molecules, and gene-regulated processes.

In an exemplary aspect, genetic/bio-technical light-emitting reporters that propagate with the labeled cells as they multiply in a subject can be used. For example, luciferases and fluorescent proteins are two common genetic markers. The luciferase enzyme, when combined with the substrate luciferin, oxygen, and ATP, generates light through a chemical reaction, resulting in bioluminescence. Genetic light-emitting reporters can be integrated into a subject and expressed using methods known to those of skill in the art. In the case of fluorescent proteins, desired targets can be illuminated with an excitation source in order to fluoresce.

In a further aspect, bioluminescence in mammalian cells can be, for example, accomplished by incorporating the luc gene into a cell's DNA in order to express the luciferase enzyme. The substrate luciferin can be added exogenously and distributes throughout the subject. The luc gene originates from the North American firefly, Photinus pyralis, and produces light at an emission peak at 560 nm. The luciferase lux gene from soil bacterium (Photorhabdus luminescens) along with substrate-encoding genes can be incorporated into a targeted cell so that both luciferase and luciferin are produced endogenously. The firefly luciferase has a very broad spectrum and contains a large component above 600 nm where transmission through tissue is higher.

Two common fluorescent proteins are green fluorescent protein and red fluorescent protein or DsRed. Bioluminescence has an advantage over fluorescence as an in vivo reporter in that no external light source is required for excitation, resulting in improved signal-to-noise ratio as compared to that with fluorescence. In the case of fluorescence, the signal level is related to both the number of cells and the intensity of excitation light.

A subject's cells can be engineered to express, or be labeled with, a fluorescent and/or bioluminescent marker using methods known in the art. Exemplary methods of labeling targets with fluorescent and/or bioluminescent markers are described in the following references and other methods are known to those skilled in the art. See, C. H. Contag and M. H. Bachmann, “Advances in vivo bioluminescence imaging of gene expression,” Annual Review Of Biomedical Engineering, vol. 4, pp. 235-260, 2002, which is incorporated herein in its entirety by reference for the methods taught therein. See also, B. W. Rice, M. D. Cable, and M. B. Nelson, “In vivo imaging of light-emitting probes,” J. Biomed. Opt., vol. 6, pp. 432-440, 2001; V. Ntziachristos, C. H. Tung, C. Bremer, and R. Weissleder, “Fluorescence molecular tomography resolves protease activity in vivo,” Nature Medicine, vol. 8, no. 7, pp. 757-760, July 2002; and C. Bremer, V. Ntziachristos, and R. Weissleder, “Optical-based molecular imaging: contrast agents and potential medical applications,” Eur Radiol, vol. 13, no. 2, pp. 231-43, 2003, which are also incorporated herein in their entirety for the methods taught therein.

In one aspect, an optical data receiving mechanism can be inserted into a subject to collect signal data from a bioluminescent or fluorescent light source. Measurements can be taken on a light-collecting surface or mechanism of the optical data receiving mechanism. It is contemplated that measurements or data collection can be performed once or at multiple times at a given location in a subject, or as described below, at multiple locations within the subject. Moreover, such data collection can be performed once or multiple times at each location of the optical data receiving mechanism.

In a further aspect of the invention, the collected data can be transferred or transmitted into a computational device for processing and processed therein to obtain quantitative information about the source including the center, total energy, and/or an estimate of the absorption/scattering properties of the underlying body region of interest. In another aspect, features of the source intensity distribution can be extracted, estimated, determined, or reconstructed using algorithms. In one aspect, mathematically, the computational algorithms can be solved completely or partially as an inverse optical source problem.

Thus, an exemplary method of computing parameters or features of a light source located within a subject comprises acquiring light source signal data from the source. Signal data from the subject's tissue, “tissue signal data,” can also optionally be acquired. As used herein light source signal and tissue signal data can be referred to as “optical data.” In one aspect, acquisition can comprise capturing light source signal data and/or tissue signal data using an optical data receiving mechanism. In this aspect, the optical data receiving mechanism is positioned and receives the light source data signal and/or tissue signal data at a location within the subject.

Light signal data and/or tissue signal data can be transmitted from the optical data receiving mechanism to a signal detection device along with associated needle positional and subject anatomical information. Transmission can be accomplished using methods known in the art, for example, by fiber optic means. For example, See, U. Utzinger and R. R. Richard-Kortum, “Fiber optic probes for biomedical optical spectroscopy,” Journal Of Biomedical Optics, vol. 8, no. 1, pp. 121-147 (2003), which is incorporated for the methods taught therein. Tissue signal data from the subject's tissue can be acquired subsequent to contacting the tissue with light energy transmitted by a laser or other light source.

The optical data receiving mechanism can be positionally moveable about and between a first location within the subject and at least one subsequent location within the subject. Thus, after acquiring light source data and/or tissue signal data at the first location, the optical data receiving mechanism can be positionally moved to at least one subsequent location within the subject, and light source signal data and/or tissue signal data can be acquired at least at the one subsequent location. In one exemplary aspect, the light source signal data and/or tissue signal data acquired within the subject can be transmitted from the optical data receiving mechanism to a computational device, and the transmitted data can be used to compute or estimate the source parameters/features and/or the source distribution can be reconstructed using an estimation and/or reconstruction algorithm(s).

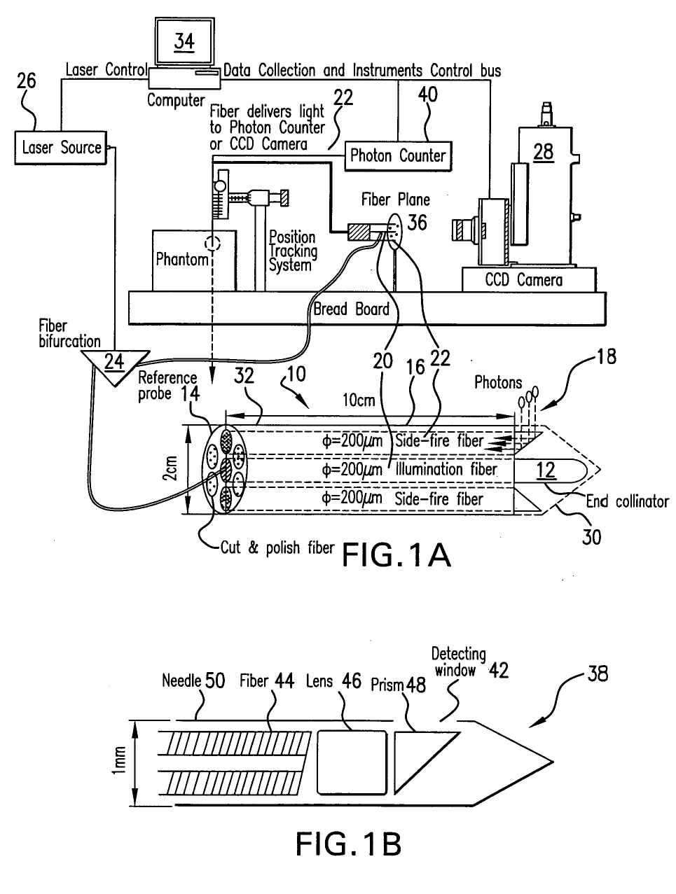

FIG. 1 A is a schematic diagram showing an exemplary optical data receiving mechanism and exemplary components of an exemplary computational optical biopsy system. As shown in FIG. 1A, the optical data receiving mechanism 10 can comprise a first end 12, a spaced second end 14 and a body portion 16 positioned therebetween. At least the first end and a portion of the body can be positioned within the subject along a track. By “a track” is meant a trajectory of the optical data receiving mechanism within the subject. At least a portion 18 of the optical data receiving mechanism positioned within the subject along the track can be configured to receive optical data. At least the first end and at least a portion of the body of the optical data receiving mechanism can also be positioned along one or more subsequent tracks within the subject. The light source data and/or the tissue signal data can be acquired from at least one location that is located along or neighboring to at least one track of the optical data receiving mechanism. FIG. 1B is a schematic diagram showing an exemplary optical data receiving mechanism 38. The optical data receiving mechanism 38 can comprise a detecting window 42. The optical data receiving mechanism 38 can comprise at least one a detection fiber 44, a collimating lens 46 and a micro-prism 48 attached with optically transparent, biocompatible cement to form a single unit and inserted into a thin-wall hypodermic stainless-steel needle 50. Because of its smaller diameter, the optical data receiving mechanism 38 can be rotated with minimal drag while it is in contact with the tissue. The optical data receiving mechanisms can be used with an illumination fiber that is separate from the receiving mechanism itself. A light source located in a subject can be detected by rotating and moving the optical data receiving mechanism 38 or 10.

In one aspect, the optical data receiving mechanism positioned within the subject along one or more tracks can be configured to transmit photons of light near or in contact with the light source located within the subject. An optical data receiving mechanism can be configured to transmit photons of light when the light source to be detected is a fluorescent light source. In is contemplated that an optical data receiving mechanism that transmits photons can be used with any light source, however, such as near or within a bioluminescent source region to measure the optical properties of the background tissue. Thus, as described above, a laser light source can be used when optical data from the subject's tissue is acquired.

The optical data receiving mechanism can comprise a commercial breast biopsy needle assembly (SenoRx Inc, CA) that is modified for use with the disclosed systems and methods or a dedicated biopsy assembly for a specific application. Optionally, the commercial biopsy device is adapted to house one fiber for illumination (“source fiber”) 20 and six fibers 22 for detection. If light transmission is not used in the biopsy needle, however, the biopsy device can comprise fibers for detection only. The excitation light can be provided by an external light source in the fluorescent imaging case or may not be used in the bioluminescent imaging case. As would be clear to one skilled in the art, however, other combinations of source fiber(s) 20 and detection fiber(s) 22 can be used, as can other optical path designs.

Optionally, the core diameter and total length of each fiber are about 200 μ and 150 cm, respectively. In this aspect, the fibers can be coated with black coating material to avoid crosstalk between them. In another aspect, the fibers can have silicone-coated monocoil jacketing with dacron braid to protect them and provide strain relief. If illumination capabilities are desired, the illuminating end of the source fiber can be cut and polished to deliver collimated or less collimated light at the tissue location of interest.

The source fiber 20 can be bifurcated by a fiber bifurcation device 24 such that one path is connected from a light source 26 to the optical data receiving mechanism 10 and the other to a signal detection device 28, for example a CCD camera, for measurement of the incident flux, or to a photon counter 40. The detector fibers 22 can be in contact with the tissue, optionally four of which can have side-fired tips while the other two can have cut and polished tips to collect optical information from any direction. The fibers can also be covered with material that allows for the passing of light energy therethrough the material. The signal detection device 28 can transmit data for processing to a computational device, for example, a high end computer 34.

The optical data receiving mechanism surface parts in direct contact with the tissue can be covered with biocompatible material. The fibers can protrude out the first end of the optical data receiving mechanism and can be capped with glass 30. This fiber assembly can be coaxially placed inside a cannula 32. The other ends of the fibers can be terminated on a fiber plate or plane 36 for an appropriate spatial arrangement in front of the signal detection system.

The hardware components and system assembling of the optical data receiving mechanism can be made using materials and methods known in the art, such as those available from Polymicro Technologies Inc. (Phoenix, Ariz.). If a source fiber is used, a fiber coupled laser system of wavelengths suitable for stimulating fluorescence or measuring tissue properties of interest, such as about 650 nm, or an interval in a near infrared range, can be used as an excitation source for the light source within the subject (Edmund Optics, NJ).

A CCD camera is one non-limiting example of a photon detector that can be used as the signal detector to capture an image of the detector fibers while acquiring an image of the illuminating reference source fiber. Received diffuse signals through the detector fibers can be collected and recorded on the CCD camera for a period of time, such as a few mille-seconds to minutes. The CCD camera can also be used to detect bioluminescent signals through the detector fibers without laser excitation. A photon counter can also be used.

When weak light signals are being detected, the camera can be cooled to reduce the background noise level. The detectors accumulate electrons knocked free by incident photons, but the incoming light of interest is not the only source to create charges in a pixel well. Because the detectors are sensitive to heat, even in total darkness a detector may record thermally induced charges, yielding a so-called dark signal (usually measured in electrons per pixel per hour). Typically, the higher the temperature is, the stronger the dark signal becomes. For example, the dark signal doubles approximately every 6° C., and may compromise the signal-to-noise ratio in low light level applications.

A back-illuminated and high-performance CCD camera can be used (Princeton Instruments, AZ) and allows permanent vacuum and deep cooling, and can be cooled down to −110° C. by liquid nitrogen for low dark charge. The dark current is 0.5e −/p/hr @ −110° C. For example, an array of 10×10 cells, each of which has 20 μm diameter can be effectively detected. These cells are within the field of view of a 200 μm diameter fiber, since numerical aperture of the fiber (NA 0.22) well covers the cell array. Each cell may emit 5-100 photons/s into 4π steradians (Rice, Cable and Nelson 2001). The total number of emitted photons collected by the fiber is about 250-5000. Assuming a unit magnification factor, each 20 μm by 20 μm detector pixel receives 3-60 photons/s, which is significantly higher than the dark signal per pixel. For the optical data receiving mechanism 38, the single photon counting module (PerkinElmer Inc.) can be utilized. It has a quantum efficiency about 65% at the wavelength 650 nm, the dark count less than 10 counts/second (5-40 0C.), and the detection frequency 20 MHz (2×10⁷ photons/second). In practically meaningful cases (>10⁵ cells in a source), the optical data receiving mechanism 38 can easily the light source in its neighborhood within about one second, with the signal outperforming the dark count (˜10 counts/second). When the source is weak and deep (for example, ˜5000 cells in a source), the light signal may not be detectable on the external surface of a living subject. Such a source can be sensed using the optical data receiving mechanism 38.

The camera can be calibrated using methods known in the art, for example, as described by Rice, Cable and Nelson (2001), In vivo imaging of light-emitting probes, J. Biomed. Optics 6(4): 432-440, which is incorporated for the methods taught therein. An 8-inch integrating sphere from Sphere Optics (LR-8-LC Low level 8″ radiant source system), which uses a night vision monitor resolving 10e-7 F-L or equivalent can be employed. The sphere can be illuminated with a tungsten lamp. A filter selects a particular wavelength with FWHM 20 nm. A variable attenuator can control light level entering the sphere. For a selected wavelength, gray levels can be correlated to intensity measures. Then, a calibration formula can be established at that wavelength for the CCD camera.

Image processing can also be used to suppress data noise and compensate for out-of-focus effects. In one example, the photon noise model is approximately a Poisson process. The out-of-focus blurring can be experimentally measured. It is contemplated that conventional denoising and deblurring algorithms can be applied.

In a further aspect, the 3D location of the optical data receiving mechanism or biopsy needle positioned within the subject that is configured to receive the light source signal data and/or tissue signal data can be monitored. Optionally, monitoring can comprise visualizing at least a portion of the optical data receiving mechanism that is located within the subject using an imaging technology selected from the group consisting of wireless positioning, ultrasound, computer tomography, magnetic resonance imaging, and others. For example, monitoring can comprise a positional and/or anatomical data receiving mechanism located on specific portion(s) of the optical data receiving mechanism that is located within and/or outside the subject.

Thus, in exemplary aspects, the optical data receiving mechanism can be positioned at least at one subsequent location and signal source data and/or tissue signal data, anatomical data from the subject, and positional data from the optical data receiving mechanism located within the subject at least at the one subsequent location can be acquired. At least a portion of the acquired data can be transmitted to a computational device, and at least a portion of the transmitted signal data can be used to determine the distribution of the light source or a feature of the light source.

Specifically, the disclosed systems, apparatuses and methods, can be used in combination with another imaging modality such as ultrasound imaging so that the biopsy needle can be guided in a transparent environment, and the computation can utilize the positional and anatomic information derived from ultrasound imaging.

Thus, the optical data receiving mechanism can be guided and monitored by an ultrasound scanner to probe a region of interest along single or multiple trajectories or tracks. Using, for example, ultrasound imaging, not only can the relative positions between the track and regions of interest be determined, but also relative configurations of the needle trajectories can be determined. Ultrasound guided interventions, such as needle biopsy for renal, pancreatic, liver, thyroid, and breast masses are known in the art. The sound waves make echoes that reflect the tissue heterogeneity, and can be reconstructed into tomographic videos depicting 2D/3D/4D images. The biopsy needle insertion trajectories can be related to structural landmarks in the subject, and can be guided through regions of interest within the subject. The 3D configurations of the needle trajectories in relation to structural/anatomical features can be used as a geometric framework. Along these trajectories, corresponding profiles of source signal intensities, optionally along with profiles of tissue properties, are measured or estimated locally. All these data can be processed and analyzed to recover the source distributions and their features.

Acquired light source signal data and/or tissue signal data can be transmitted from the optical data receiving mechanism to a computational device. Thus, a computational device can be configured to receive data acquired using the optical data receiving mechanism.

The fluorescent and/or bioluminescent parameters, such as source center, total energy, and other features, can be estimated or reconstructed from the transmitted source signal data and the parameters can be coupled with the associated anatomical and positional information. The source parameters/features and/or source distribution can be computed using an estimation and/or reconstruction algorithm(s), which use the relevant data.

While the radiative transport equation (RTE) or its variant versions can be used to serve as the forward model for the computational optical biopsy (COB), a stationary diffusion approximation can also be used as the forward model. For example a stationary diffusion approximation model can be used in the case of bioluminescent imaging. The case of fluorescent imaging can be discussed similarly with the addition of the light excitation related terms and other details. Hence, the forward model for the light flux induced from the internal bioluminescence source qo is modeled by the following partial differential equation and the decay condition at infinity:

$$\begin{matrix}\begin{matrix}- ∇ \cdot ( D ∇ \cdot {u}_{0} ) + {μ}_{a} {u}_{0} = {q}_{0} , & x ∈ {R}^{3} ,\end{matrix} & ( 1 ) \\ {lim}_{x \to ∞} {u}_{0} ( x ) = 0. & ( 2 )\end{matrix}$$

As an approximation, the light distribution after the insertion of the biopsy needle or optical data receiving mechanism is considered the same as it was before insertion. More accurate modeling on the effect of the insertion of the biopsy needle also can be performed. Because there is no incoming light during measurement from the optical data receiving mechanism, the boundary condition at the probe exposure surface under the diffusion approximation can be prescribed as

$${u}_{0} ( x ) + 2 D ( x ) \frac{\partial {u}_{0}}{\partial v} ( x ) = 0. \qquad ( 3 )$$

The measurement from the exposure surface of the optical data receiving mechanisms 10 and 38 is given by

$$\begin{matrix}g ( x ) = - D ∇ {u}_{0} ( x ) \cdot v ( for 10 ) \\ {g}_{point}^{measure} = \frac{{q}_{0} {ⅇ}^{- {μ}_{eff} x} {F}^{2} [ x {sin}^{2} ( α ) + 2 D ( 1 - {cos}^{3} ( α ) ) cos ( σ ) ( 1 + x {μ}_{eff} ) ]}{16 D {x}^{2}} ( for 38 )\end{matrix}$$

where x is the measurement point and v is the normal of the optical data receiving mechanism. σ is the angle between the vectors r and v, r is the position vector, α is the half maximum incident angle of photons detectable on the needle detecting window 38, which is related to the needle probe numerical aperture NA=sin α, F is the radius of the detecting window. The effects of a limited numerical aperture of the optical data receiving mechanism and different refraction indices at the exposure surface using the established methods known in the field (See, R. C. Haskell, L. O. Svaasand, T. T. Tsay, T. C. Feng, M. S. McAdams, and B. J. Tromberg, “Boundary conditions for the diffusion equation in radiative transfer,” Journal of the Optical Society of America, A, vol. 11, no. 10, pp. 2727-2741, October 1994; U. Utzinger and R. R. Richard-Kortum, “Fiber optic probes for biomedical optical spectroscopy,” Journal Of Biomedical Optics, vol. 8, no. 1, pp. 121-147, 2003). By (3) and (4), the measurement obtained with the optical data receiving mechanism is approximately given by

$$m ( x ) = \frac{1}{2} {u}_{0} ( x ) \qquad ( 5 )$$

where x is the position of the optical data receiving mechanism.

The forward solution is found for sources of the following form (See, G. Wang, Y. Li, and M. Jiang, “Uniqueness theorems for bioluminescent tomography,” Medical Physics, vol. 31, no. 8, pp. 2289-2299, 2004):

$${q}_{0} ( y ) = {\sum }_{k = 1}^{K} {q}_{k} {Λ}^{k} {χ}_{B ( {ξ}_{k} ; {R}_{k} )} ( y ) , \qquad ( 6 )$$

where B(ξₖ;Rₖ) is a ball with the center at ξₖ and radius Rₖ, and Λ^{k} is the source intensity on the ball. Sources of the above form have been studied (Wang, et al. (2004)) for bioluminescence tomography and constitutes a class of so-called radial base functions, which can approximate any source function effectively. (See, M. D. Buhmann, Radial basis functions: theory and implementations, ser. Cambridge Monographs on Applied and Computational Mathematics. Cambridge: Cambridge University Press, 2003, vol. 12).

Assume that there is only one light source q₀=Λ_{χB}(ξ;R), where B(ξ;R) is the ball centered at ξ=(X, Y, Z) with radius R>0. Then, the solution to (1) with such D, μₐ and q₀(y), which decays at ∞, can be obtained as follows.

The origin is translated to ξ and then the translated solution is radially symmetrical. Hence, for r=∥y-ξ∥<R,

$$- D \frac{{ⅆ}^{2} {u}_{0}}{ⅆ {r}^{2}} ( r ) - \frac{2 D}{r} \frac{ⅆ {u}_{0}}{ⅆ r} ( r ) + {u}_{0} ( r ) {μ}_{a} = Λ . \qquad ( 7 )$$

Then, the solution is given by

$${u}_{0} ( r ) = C \frac{sinh ( \sqrt{\frac{{μ}_{a}}{D}} r )}{r} + \frac{Λ}{{μ}_{a}} , \qquad ( 8 )$$

where C is an arbitrary constant to be determined later. Next, to find the solution outside the light source the following equation is solved:

$$- D \frac{{ⅆ}^{2} {u}_{0}}{ⅆ {r}^{2}} ( r ) - \frac{2 D}{r} \frac{ⅆ {u}_{0}}{ⅆ r} ( r ) + {μ}_{a} {u}_{0} ( r ) = 0 , \qquad ( 9 )$$

by matching the u₀(R) and ú₀(R) from the boundary of the light source, i.e., using

$${u}_{0} ( R ) = C \frac{sinh ( \sqrt{\frac{{μ}_{a}}{D}} R )}{R} + \frac{Λ}{{μ}_{a}} , \\ {u}_{0}^{′} ( R ) = - C \frac{sinh ( \sqrt{\frac{{μ}_{a}}{D}} R )}{{R}^{2}} + C \sqrt{\frac{{μ}_{a}}{D}} \frac{cosh ( \sqrt{\frac{{μ}_{a}}{D}} R )}{R} , \qquad ( 10 )$$

and obtain that

$$\frac{{ⅇ}^{- \sqrt{\frac{{μ}_{a}}{d}} r - \sqrt{\frac{{μ}_{a}}{D}} R}}{2 {μ}_{a} \sqrt{\frac{{μ}_{a}}{D}} r} ( {ⅇ}^{2 \sqrt{\frac{{μ}_{a}}{D}} r} Λ - {ⅇ}^{2 \sqrt{\frac{{μ}_{a}}{D}} R} Λ + {ⅇ}^{2 \sqrt{\frac{{μ}_{a}}{D}} r} Λ \sqrt{\frac{{μ}_{a}}{D}} R + {ⅇ}^{2 \sqrt{\frac{{μ}_{a}}{D}} R} Λ \sqrt{\frac{{μ}_{a}}{D}} R + C {ⅇ}^{2 \sqrt{\frac{{μ}_{a}}{D}} r} {μ}_{a} cosh ( \sqrt{\frac{{μ}_{a}}{D}} R ) - C {ⅇ}^{2 \sqrt{\frac{{μ}_{a}}{D}} R} {μ}_{a} cosh ( \sqrt{\frac{{μ}_{a}}{D}} R ) + C {ⅇ}^{2 \sqrt{\frac{{μ}_{a}}{D}} r} {μ}_{a} sinh ( \sqrt{\frac{{μ}_{a}}{D}} R ) + C {ⅇ}^{2 \sqrt{\frac{{μ}_{a}}{D}} R} {μ}_{a} sinh ( \sqrt{\frac{{μ}_{a}}{D}} R ) ) .$$

To have such a solution decay at ∞, the coefficient in front of the exponential term

$${ⅇ}^{\sqrt{\frac{{μ}_{a}}{D}} r}$$

must be zero. Therefore,

$$C = - \frac{Λ ( 1 + \sqrt{\frac{{μ}_{a}}{D}} R )}{{ⅇ}^{\sqrt{\frac{{μ}_{a}}{D}} R} {μ}_{a}} \qquad ( 11 )$$

which finalizes the solution as follows.
For r<R

$${u}_{0} ( r ) = \frac{Λ}{{μ}_{a}} - \frac{Λ ( 1 + \sqrt{\frac{{μ}_{a}}{D}} R ) sinh ( \sqrt{\frac{{μ}_{a}}{D}} r )}{{ⅇ}^{\sqrt{\frac{{μ}_{a}}{D}} R} {μ}_{a} \sqrt{\frac{{μ}_{a}}{D}} r} \qquad ( 12 )$$

and for r≧R,

$$\begin{matrix}{u}_{0} ( r ) = \frac{Λ ( 1 + \sqrt{\frac{{μ}_{a}}{D}} R + {ⅇ}^{2 \sqrt{\frac{{μ}_{a}}{D}} R} ( - 1 + \sqrt{\frac{{μ}_{a}}{D}} R ) )}{2 {ⅇ}^{\sqrt{\frac{{μ}_{a}}{D}} ( r - 1 - R )} {μ}_{a} \sqrt{\frac{{μ}_{a}}{D}} r} & ( 13 ) \\ = \frac{Λ}{2 {μ}_{a \sqrt{\frac{{μ}_{a}}{D}} r}} ( [ 1 + \sqrt{\frac{{μ}_{a}}{D}} R ] {ⅇ}^{- \sqrt{\frac{{μ}_{a}}{D}} R} + [ - 1 + \sqrt{\frac{{μ}_{a}}{D}} R ] {ⅇ}^{\sqrt{\frac{{μ}_{a}}{D}} R} ) {ⅇ}^{- \sqrt{\frac{{μ}_{a}}{D}} r} & ( 14 ) \\ = \frac{Λ}{{μ}_{a \sqrt{\frac{{μ}_{a}}{D}} r}} ( \sqrt{\frac{{μ}_{a}}{D}} R cosh ( \sqrt{\frac{{μ}_{a}}{D}} R ) - sinh ( \sqrt{\frac{{μ}_{a}}{D}} R ) ) {ⅇ}^{- \sqrt{\frac{{μ}_{a}}{D}} r} & ( 15 )\end{matrix}$$

where φ(r) is used to denote the unique positive radial solution of

$$\begin{matrix}- D Δφ + {μ}_{a} φ = - D ( {φ}^{″} + \frac{N - 1}{τ} {φ}^{′} ) + {μ}_{a} φ = 0 , & ( 16 ) \\ φ ( 0 ) = 1 , and {φ}^{′} ( 0 ) = 0. & ( 17 )\end{matrix}$$

It can be solved as

$$φ ( r ) = \frac{sinh ( \sqrt{\frac{{μ}_{a}}{D}} r )}{\sqrt{\frac{{μ}_{a}}{D}} r} . \qquad ( 18 )$$

Its solid integral inside the source support B(ξ;R) is defined to be the weighted-moment:

$$\begin{matrix}M ( Λ , R ) = Λ {\int }_{0}^{R} 4 π {r}^{2} ϕ ( r ) ⅆ r , & ( 19 ) \\ = \frac{4 D Λπ ( {μ}_{a} R cosh ( \sqrt{\frac{{μ}_{a}}{D}} R ) - \sqrt{{μ}_{a} D} sinh ( \sqrt{\frac{{μ}_{a}}{D}} R ) )}{{μ}_{a}^{2}} & ( 20 )\end{matrix}$$

Therefore, the solution outside the light source is equal to

$$\begin{matrix}{u}_{0} ( r ) = \frac{Λ}{{μ}_{a \sqrt{\frac{{μ}_{a}}{D}} r}} ( \sqrt{\frac{{μ}_{a}}{D}} R cosh ( \sqrt{\frac{{μ}_{a}}{D}} R ) - sinh ( \sqrt{\frac{{μ}_{a}}{D}} R ) ) {ⅇ}^{- \sqrt{\frac{{μ}_{a}}{D}} r} & ( 21 ) \\ = \frac{Λ}{{μ}_{a \sqrt{\frac{{μ}_{a}}{D}} r}} \frac{1}{\sqrt{{μ}_{a} D}} \frac{{μ}_{a}^{2}}{4 D Λπ} M ( Λ , R ) {ⅇ}^{- \sqrt{\frac{{μ}_{a}}{D}} r} & ( 22 ) \\ = \frac{1}{4 π D r} M ( Λ , R ) {ⅇ}^{- \sqrt{\frac{{μ}_{a}}{D}} r} . & ( 23 )\end{matrix}$$

Let

$${μ}_{eff} = \frac{{μ}_{a}}{D}$$

be the effective attenuation coefficient. (See, W. F. Cheong, S. A. Prahl, and A. J. Welch, “A review of the optical properties of biological tissues,” IEEE Journal of Quantum Electronics, vol. 26, pp. 2166-2185, December 1990). The weighted-moment Λ_{eff}=M (Λ, R) is called the effective intensity of the ball source and denoted by Λ_{eff}. The final solution of the light flux at any location χ for any source center ξ is given by the following formula:

$${u}_{0} ( x ) = \{ \begin{matrix}\frac{Λ}{{μ}_{a}} - \frac{Λ ( 1 + \sqrt{{μ}_{eff}} R ) sinh ( \sqrt{{μ}_{eff}} r )}{{ⅇ}^{\sqrt{{μ}_{eff}} R} {μ}_{a \sqrt{{μ}_{eff}} r}} , & r < R , \\ \frac{M ( Λ , R )}{4 π D r} {ⅇ}^{- \sqrt{{μ}_{eff}} r} , & r \geq R ,\end{matrix} \qquad ( 24 )$$

where r=∥x-ξ∥ is the distance from an arbitrary point x to the source center ξ.
Solution for a Point Source

The forward solution for a point source with intensity Λ is:

$${u}_{0} ( x ) = \frac{Λ}{4 π D r} {ⅇ}^{- \sqrt{{μ}_{eff}} r} , r > 0. \qquad ( 25 )$$

For point sources, the effective intensity Λ_{eff} is equal to its intensity Λ.
Solution for Multiple Ball and Point Sources

For a general source as in (6) consisting of multiple ball or point sources (when Rₖ=0), by (24) and (25), the forward solution generated by those sources outside the source support can be given by

$${u}_{0} ( x ) = {\sum }_{k = 1}^{K} \frac{{Λ}_{eff}^{( k )}}{4 π D {r}_{k}} {ⅇ}^{- \sqrt{{μ}_{eff}} {r}_{k}} , \qquad ( 26 )$$

where r=∥x−ξₖ∥, for k=1, . . . K.

Let
x(t)=(o₁+n₁t, o₂+n₂t, o₃+n₃t), (27)
be the parametric form of the insertion path where n=[n₁, n₂, n₃] (28)
is the insertion direction of the biopsy needle path. By (5), for a source consisting of multiple ball and point-sources, the measurement at one point x(t) on the insertion path outside the source support can be approximated by

$$m ( x ( t ) ) = \frac{1}{2} {u}_{0} ( x ( t ) ) = {\sum }_{k = 1}^{K} \frac{{Λ}_{eff}^{( k )}}{8 π D {r}_{k}} {ⅇ}^{- \sqrt{{μ}_{eff}} {r}_{k}} . \qquad ( 29 )$$

Multiple insertions generate more measurement equations as the above. Assuming that the measurement can be conducted at M points along multiple insertion paths, the measurement equations are arrived at,

$${m}_{i} = {\sum }_{k = 1}^{K} \frac{{Λ}_{eff}^{( k )}}{8 π D {r}_{i , k}} {ⅇ}^{- \sqrt{{μ}_{eff}} {r}_{k}} . \qquad ( 30 )$$

where r_{i,k}=∥xᵢ−ξₖ with xᵢ=(xᵢyᵢ, zᵢ) being the i-th measurement point on one insertion path, for i=1, . . . , M, and ξₖ=(Xₖ; Yₖ;Zₖ) being the k-th source center, for k=1; . . . , K. Let

$${\overline{Λ}}^{( k )} = \frac{{Λ}_{eff}^{( k )}}{8 π D} , and \overline{μ} = \sqrt{{μ}_{eff}} . \qquad ( 31 )$$

The idea is to use the measurement equations to solve the effective attenuation coefficient μ_{eff}, the rescaled source effective intensities Λ̅_{eff}^{(k)} and the source centers ξₖ=(Xₖ, Yₖ, Zₖ). There are totally 1+K+3K=4K+1 unknowns, 1 parameter μ_{eff}, K effective intensity values Λ_{eff}^{(k)} and 3K center coordinates. If the number M of measurement is greater than 4K+1, it is possible to find μ_{eff}, Λ_{eff}^{(k)} and ξₖ by solving or fitting the following equations: (If the parameter D and/or μ_{eff} is found with other techniques, then the real source effective intensity Λ^{(k)} can be obtained.)

$${\sum }_{k = 1}^{K} {\overline{Λ}}^{( k )} \frac{{ⅇ}^{- \overline{μ} {r}_{i , k}}}{{r}_{i , k}} = {m}_{i} , \qquad ( 32 )$$

for i=1, . . . , M.

The parameters μ_{eff}, Λ̅_{eff}^{(k)} and ξₖ can be reconstructed by solving (32) with a nonlinear least-squares fitting technique, (See, J. E. J. Dennis, “Nonlinear least-squares,” in The state of the art in numerical analysis: proceedings of the Conference on the State of the Art in Numerical Analysis, D. A. H. Jacobs, Ed. London; New York: Academic Press, 1977), implemented in MatLab® (Natick, Mass.) for fast prototyping. Other methods based on statistical techniques such as the EM method (See, G. J. McLachlan and T. Krishnan, The EM algorithm and extensions, ser. Wiley Series in Probability and Statistics: Applied Probability and Statistics. New York: John Wiley & Sons Inc., 1997), can also be used.

The computed source parameters/features can be the intensity of the light source, the anatomical location of the light source within the subject, and other features that are calculable when the biopsy trajectories go both outside and inside the sources. Other estimation/reconstruction algorithms can be used that can be based on forward models more accurate than the diffusion equation. Also, the methods can be extended to utilize multispectral and/or dynamic optical signals for estimation/reconstruction of the underlying source parameters/features/distributions.

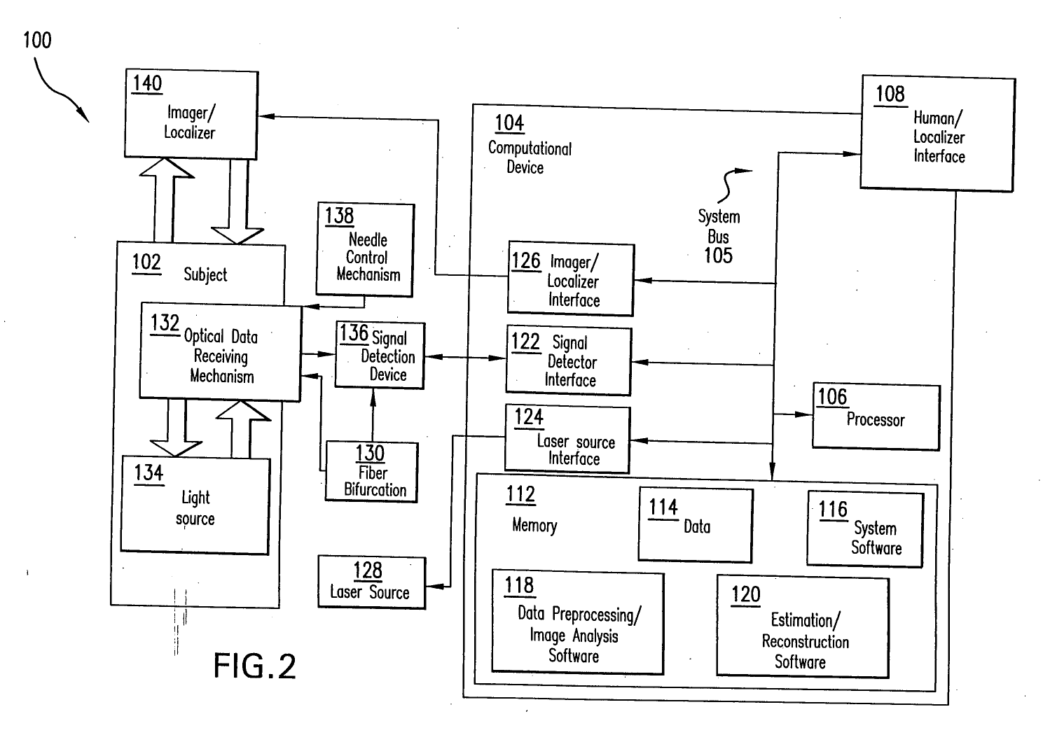

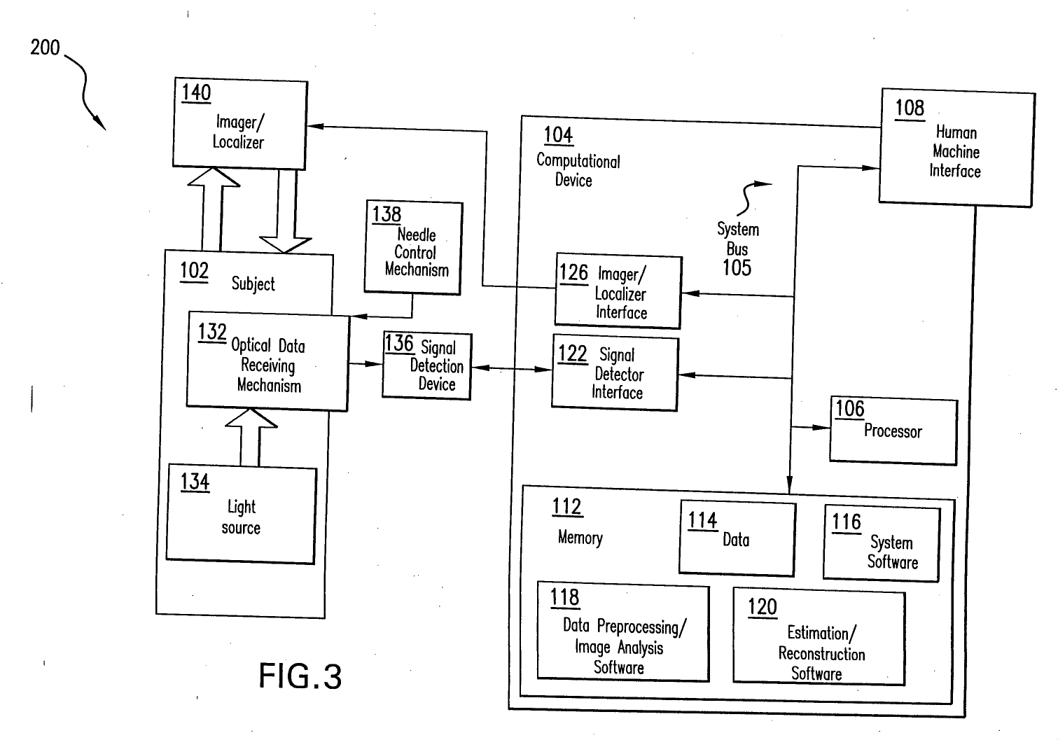

FIG. 2 is a block diagram showing an exemplary computational optical biopsy system based on fluorescence 100. FIG. 3 is a block diagram showing an exemplary computational optical biopsy system based on bioluminescence 200. These exemplary optical biopsy systems are only examples and are not intended to suggest any limitations on one or combination of components listed in the exemplary systems. These systems can be used to perform the above-described methods for computing source parameters of a light source within a subject.

Referring to FIGS. 2 and 3, the computational optical biopsy systems 100 and 200 can operate on a subject 102. As described above, the subject 102 can be a small animal, such as a mouse, rat or rabbit. The subject can also be a primate, including a human. Thus, the term “subject” is not intended to be limited to any particular species. Moreover, the term is not limited to any gender or age, and fetuses and embryos are also included within the definition of “subject” 102.

The computational optical biopsy systems 100 and 200 comprise a computational device 104. As would be clear to one skilled in the art, any common computational device could be used, such as a computer. The computational device 104 can also comprise multiple computational devices networked together using methods, software and hardware as would be known to one skilled in the art. Thus, although the computational device is used in its singular form, embodiments are included wherein multiple computational devices operating in a networked environment using logical connections to one or more remote computing devices. By way of example, a remote computing device can be a personal computer, portable computer, a server, a router, a network computer, a peer device or other common network node, and the like.

Logical connections between the computational device and a remote computing device can be made via a local area network (LAN) or a general wide area network (WAN) or other means. Such networking environments are commonplace in offices, enterprise-wide computer networks, Intranets, and the Internet. In a networked environment, data preprocessing/image analysis software 118, estimation/reconstruction software 120 and system software 116 (including an operating system), and data 114, or portions thereof, can be stored in a remote memory storage device (not shown). For purposes of illustration, application programs and other executable program components such as the system software are illustrated herein as discrete blocks, although it is recognized and contemplated that such programs and components reside at various times in different storage components of the computational device, and are executed by the processor(s) of the computational device.

In one aspect, the computational device 104 further comprises a system bus 105. In this aspect, the system bus couples or connects the various components of the computational device including a human/machine interface 108, a processor 106, a memory 112, a laser source interface 124, a signal detector interface 122 and an imager/localizer interface 126.

Of course, the system bus 105 represents one or more of several possible types of bus structures, including a memory bus or memory controller, a peripheral bus, an accelerated graphics port, and a processor or local bus using any of a variety of bus architectures. By way of example, such architectures can include an Industry Standard Architecture (ISA) bus, a Micro Channel Architecture (MCA) bus, an Enhanced ISA (EISA) bus, a Video Electronics Standards Association (VESA) local bus, and a Peripheral Component Interconnects (PCI) bus also known as a Mezzanine bus. This bus, and all buses specified in this description can also be implemented over a wired or wireless network connection and each of the subsystems, including the processor 106, memory 112, system software 116, data 114, data preprocessing/image analysis software 118, estimation/reconstruction software 120, a laser source interface 124, a signal detector interface 122, and an imager/localizer interface 126 can be contained within one or more remote computers (not shown) at physically separate locations, connected through buses of this form, implementing a fully distributed system.

The computational device typically includes a variety of computer readable media. Such media can be any available media that is accessible by the computational device and includes both volatile and non-volatile media, removable and non-removable media.

The human/machine interface 108, which is connected with the system bus 105 allows a user to input commands or data into the computational device 104. The input data or commands can interact with the other components of the computational device 104 through connections provided by the systems. The human/machine interface 108 also functions as an interface with a user wherein the user can visualize information such as an image created by the computational optical biopsy system 100 or 200. Thus, the human/machine interface can comprise a display or other output device for visualizing an image and/or presenting extracted information.

Thus, a user can enter commands and information into the computational device via an input device. Examples of such input devices include, but are not limited to, a keyboard, pointing device (e.g., a “mouse”), a microphone, a joystick, a serial port, a scanner, and the like. These and other input devices can be connected to the processing unit 106 via a human machine interface 108 that is coupled to the system bus 105, but may be connected by other interface and bus structures, such as a parallel port, game port, or a universal serial bus (USB).

A display device can also be included as the human machine interface or a portion thereof. A display device can be connected to the human machine interface via an interface, such as a display adapter. For example, a display device can be a monitor. In addition to the display device, other output peripheral devices can include components such as speakers and a printer, which can be connected to the computational device via typical interfaces.

The display device can also be used as an interface to input data or commands from the user into the computational device using methods, software and hardware known to those skilled in the art.

The memory component 112 or “memory” includes data 114, system software 116, data preprocessing image analysis software 118, and estimation reconstruction software 120. Memory 112 can include computer-readable media in the form of volatile memory, such as random-access memory (RAM) and/or non-volatile memory such as read-only memory (ROM). Memory 112 typically contains data such as, optical data, receiving mechanism localizing data, and tissue parameter data. These data can be input into the computational device 104, as described below.

The computational device may include other removable/non-removable, volatile/non-volatile computer storage media. For example, a storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules can be stored within memory, including by way of example, system software (including an operating system), data preprocessing/image analysis software, estimation/reconstruction software, and data. Each of the system software, data preprocessing/image analysis software, and estimation/reconstruction software (or some combination thereof) may include or overlap some elements of the other software.

In one aspect, the data stored in 114 can be in a raw, unprocessed form. The data preprocessing/image analysis software 118 can convert the raw data into a form directly useable by the estimation reconstruction software 120. The data preprocessing image analysis software is known to those skilled in the art and can be used to express the raw data in appropriate physical units. The data preprocessing image analysis software also can be used to reduce noise or system bias from the data. In various aspects, after the data preprocessing/image analysis software processes the data 114, the estimation/reconstruction software 120 provides source parameters/features/distributions of the light source located within the subject 102.

In one aspect, an implementation of the data preprocessing/image analysis software and the estimation/reconstruction software may be stored on some form of computer readable media. Computer readable media can be any available media that can be accessed by a computer. By way of example, and not limitation, computer readable media can comprise “computer storage media” and “communications media.” “Computer storage media” include volatile and non-volatile, removable and non-removable media implemented in any method or technology for storage of information such as computer readable instructions, data structures, program modules, or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The processing of the data 114 can be performed by software components coupled with hardware components. The data preprocessing software and the estimation/reconstruction software may be described in the general context of computer-executable instructions, such as program modules, being executed by one or more computers or other devices. Generally, program modules include computer codes consisting of routines, objects, data structures, etc., that perform particular computational tasks or implement particular data types or complete other specific tasks. The data preprocessing/image analysis software and the estimation/reconstruction software may also be practiced in distributed computing environments where tasks are performed by remote processing devices that are linked through a communications network. In a distributed computing environment, program modules may be located in both local and remote computer storage media including memory storage devices.

In a further aspect, the computational device 104 can also include a laser source interface 124, signal detector interface 122, and an imager/localizer interface 126. The laser source interface 124 can be used when the light source 134 within the subject 102 is fluorescent or is otherwise stimulated by an external source of laser light 128. Optionally, the laser source may also be used when tissue signal data is acquired. Thus, the laser source interface 124, which can be coupled to the system bus 105 can be coupled to an external laser source 128 such that the laser source can be directed to excite a light source within the subject. Typically, a user will input the desired parameters for light source stimulation via the human/machine interface 108. Based on these input parameters, the system software 116 in conjunction with the processor 106, which is coupled to the laser source interface 124, directs the laser source 128 to provide light at the desired parameters. Thus, the connection of the laser source 128 with the laser source interface 124 allows a user to regulate functional aspects of the laser source 128 which may include, but are not limited to, power of the laser and timing of laser excitation of the light source 134.

In another aspect, the computational device 104, as described above, can also includes a signal detector interface 122. Similar to the laser source interface 124, the single detector interface 122 can be coupled to the system bus 105 and through the system bus to other components of the system, including, but not limited to, the human/machine interface 108, the processor 106, and the memory 112. The detector interface is connected to a signal detection device 136 such that the signals detected from the underlying fluorescent and/or bioluminescent light source or data from the subject's tissue through a optical data receiving mechanism 132 can be delivered to the computational device 104. Exemplary optical data receiving mechanisms are shown in FIGS. 1A and 1B.

The signal detection device 136 can deliver source signal data and/or tissue signal data to the signal detector interface for provision to the memory 112, and in particular to the memory portion 114 for data preprocessing/image analysis and estimation/reconstruction of light source parameters.

Data delivered to the signal detector interface 122 from the signal detection device 136 can be in a digital or analog form with an analog to digital conversion occurring at any point of the system 100 or 200, as would be known to one skilled in the art, including as a part of the optical data receiving mechanism 132, the signal detection device 136, the signal device interface 122, within the computational device 104 or anything therebetween. Data delivered from signal detector interface 122 through the system bus 105 to memory 112, and in particular, the memory portion 114 is typically stored in a digital form for provision to the data preprocessing software analysis 118 and the estimation/reconstruction software 120.

Optionally, the signal detector interface 122 can also direct input to the signal detection device 136. Thus, commands typically input at the human/machine interface 108, as described above, and transferred along the system bus 105 to the signal detector interface 122, in conjunction with the other components of the computational device including the processor 106 and the memory 112, can deliver input to the signal detection device 136.

In another aspect, the computational device 104 also comprises an imager/localizer interface 126 which is connected to the system bus 105 and through the system bus to other components of the computational device 104, such as, the human/machine interface 108, the processor 106, and memory 112. Furthermore, the imager/localizer interface 106, is coupled to an imager/localizer 140, which can be located external to the computational device 104. The imager/localizer interface can receive data from the imager/localizer 140 either in an analog or digital form. Operationally, data from the imager/localizer 140 is transmitted to the imager/localizer interface 126, and from the image/localizer interface through the system bus 105 to the other components of the computational device, including the human/machine interface 108, processor 106, memory 112, and within the memory 112, to the memory portion 114.

Data from the imager/localizer can be stored in the memory portion 114 in a digital form. In the case that the original data are in an analog form, there will typically be an analog to digital conversion mechanism occurring at any point, including in the imager/localizer 140, or at other points within the computational device, or at any location therebetween.

Moreover, in a further aspect, the image/localizer interface can also be configured to deliver input to the imager/localizer 140. Thus, a user can input commands or data in the human/machine interface 108 which can be transferred along the system bus to the imager/localizer interface, and from the imager/localizer interface to the imager/localizer, which is typically located external to the computational device. The imager/localizer 140, however, can also be a fully independent system in which controlling parameters are input at a separate human/machine interface. Thus, imager/localizer 140 can be a separate component in its entirety such as an ultrasound machine with its own computational control device. Such independent ultrasound or other imaging machines are known to those skilled in the art. Data from an independent imager/localizer can be input into the computational device 104, using many alternatives known to one that is skilled in the art, such as by direct connection through the imager/localizer interface 126 or from other data transfer mechanisms.

In another aspect, the computational optical biopsy systems 100 and 200 further comprise an optical data receiving mechanism 132. Two examples of exemplary data receiving mechanisms are shown in FIG. 1A and 1B.

The optical data receiving mechanism 132, is typically inserted into the subject 102 along a first track. Thus, in one example, the optical data receiving mechanism is a needle-like structure and can be referred to as “optical biopsy needle,” or “biopsy needle,” which can be inserted into the tissue of the subject and advanced through the tissue of the subject along the first track. The optical data receiving mechanism 132 can be advanced along a first track using a needle control mechanism 138. The needle control mechanism 138 can comprise manual control of the optical data receiving mechanism. Thus, a user can manually manipulate the optical data receiving mechanism 132 by advancing it into the tissue of the subject 102 along the first track. A user can also position the optical data receiving mechanism in the subject 102 along one or more additional tracks, or multiple biopsy needles can be positioned along one or more tracks. Thus, although the optical data receiving mechanism 132 can be placed within the subject and located along one track, the optical data receiving mechanism 132 can be inserted and advanced along any number of tracts within the subject.

Positioning of the optical data receiving mechanism 132 to more tracts within subject tissue can comprise redirection of the optical data receiving mechanism 132 within the subject 102. The optical data receiving mechanism can be positionally redirected within the subject tissue. The optical data receiving mechanism can also be retracted along one tract in the tissue before being re-advanced along a subsequent tract. Thus, there is no limitation to the positioning of the optical data receiving mechanism 132 within the subject, as any means of movement allowing the optical data receiving mechanism 132 to attain one or any number of tracts within the subject is covered herein.

Using the needle control mechanism 138, the optical data receiving mechanism can be directed or redirected within the subject to attain one or any number of tracts. The needle control mechanism can be manual or automated. The needle control mechanism can operate through the computational device 104 or a similar computational device in conjunction with a motorized manipulation mechanism. By placing the optical data receiving mechanism and advancing it in the subject, signal data and/or tissue signal data can be collected at one or more points along at least one tract, or more points along multiple tracts. Thus, one or more data points can be collected from a light source 134 or from the subject's tissue at one or more locations.

The optical data receiving mechanism 132, or at least a portion of it, is configured to receive light signal data from a light source 134 and to receive signal data from the subject's tissue, which is located within the subject 102. The light source 134 can be any light source capable of emitting photons of light and, in some examples, can be either a bioluminescent light source or a fluorescent light source.

When using a fluorescent light source, the optical data receiving mechanism 132 can be configured to send photons of light to excite the fluorescent light source 134 so that it generates or emits fluorescent photons. Optionally, an optical data receiving mechanism is configured to deliver light from a laser light source 128. The laser source 128 can be coupled to the computational device 104 through the laser source interface 124 to produce laser light. Laser light can be transmitted to a fiber bifurcation 130 which bifurcates the light into two distinct paths, one of which is directed to the signal detection device 136 as a reference and the other is directed to the optical data receiving mechanism or biopsy needle 132. When a photon of laser light that was directed through the optical data receiving mechanism 132 excites a light source within the subject 102 and interacts with the background tissue in the subject 102, the resultant light signal data and/or tissue signal data can be collected by the light receiving mechanism of the optical data receiving mechanism. Thus, at least a portion of the optical data receiving mechanism is configured to receive light signal data and/or tissue signal data for provision to the signal detection device and to the computational device 104.

In examples where a bioluminescent source is used, a system can be used without a light source 128, fiber-bifurcation 130, or laser light source interface 124, as shown in the exemplary system 200. In this exemplary system, the light source 134 emits photons of light without excitation by an external light source to be detected by the optical data receiving mechanism 132. Thus, one type of the optical data receiving mechanism can be considered a passive optical biopsy needle because it is configured to receive emitted photons from the light source 134.

Another type of the needle includes a portion configured to receive photons emitted from a light source, and a transmitting mechanism by which light can be transmitted through the optical data receiving mechanism into the subject 102 such that it may measure the tissue properties of the subject 102 for better estimation/reconstruction of the underlying bioluminescent source. In either case, the received light signal data and/or tissue signal data is acquired by the optical data receiving mechanism and transmitted through the optical data receiving mechanism by, for example, fiber optics, to a signal detector device 136, which is a very sensitive photon detector, which is referred to herein as a signal detector, or to the computational device. One example of a signal detector device is a CCD camera. A transmitter or transmitting means can be used to transmit at least a portion of the acquired signal data to a computational device. Optionally, the transmitter comprises a fiber optic cable. Optionally, the transmitter comprises a signal detector device. Thus, as used herein, the term “transmitter” includes components, devices, and/or means used to transmit data acquired from the optical data receiving mechanism to the computational device.

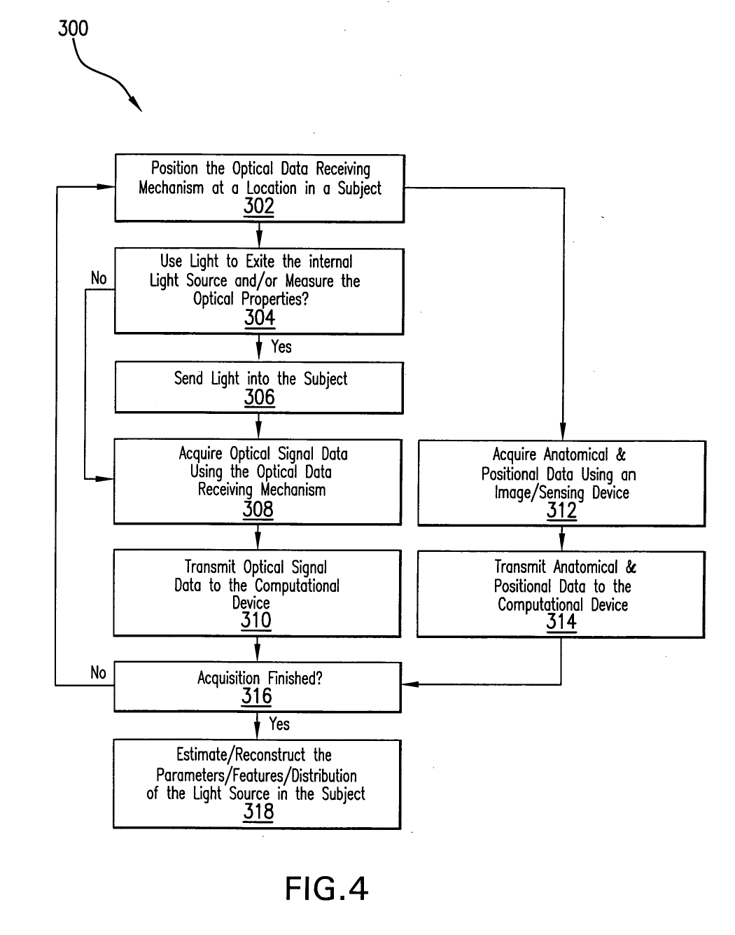

FIG. 4 is a flow chart illustrating a method 300 of computing source tissue parameters. In practice, the positionally moveable optical data receiving mechanism 132 is advanced into a subject or positioned along one tract to a location within the subject, as shown in block 302. In block 304, it is determined whether to use light to excite the internal light source and/or to measure the tissue optical properties. If it is determined that light will not be used, the optical signal data is acquired using the optical data receiving mechanism as shown in block 308. If it is determined that light will be used in block 304, then light is directed into the subject in block 306, and light source signal data and/or tissue signal data is subsequently acquired in block 308. After data is acquired in block 308, the data is transmitted to a computational device as shown in block 310. In block 312, anatomical and positional data can be acquired using an image/sensing device or estimated otherwise. The anatomical and positional data acquired in block 312 can be transmitted to the computational device in block 314. In block 316, it is determined whether the acquisition process is finished. This determination can be based on the data transmitted to the computational device in block 310 and on the data transmitted to the computational device in block 314, as well as on a variety of user input parameters. If, in block 316, it is determined that the acquisition process is finished, then the parameters, features, and/or distribution of the light source in the subject is estimated/reconstructed in block 318. If, in block 316, it is determined that the acquisition process is not finished, the optical data receiving mechanism is positioned at a subsequent location as shown in block 302.

As described above, transmitted data can be subsequently transferred into the memory of the computational device 104 and stored as data 114. The stored data can be preprocessed and analyzed using the data preprocessing/image analysis software described above.

Positional data can also be collected or acquired on the location of the optical data receiving mechanism within the subject 102 and the anatomy of the subject with the needle in place by the imager/localizer 140 in block 312. Optionally, the image/localizer 140 is an imaging modality such as an ultrasound unit, and can be a three dimensional ultrasound unit with accessories for 3D localization of the needle biopsy trajectories as correlated to the anatomy of the subject. Other imaging modalities can be used, however, such as a CT scanner or MRI scanner. For example, the optical data receiving mechanism 132 may be equipped with a transmitter(s) which transmits signals to a localizer(s) which receives data for provision to the computational device 104 for 3D localization. Thus, the imager/localizer 140 can monitor the position of the optical data receiving mechanism 132 within the subject by collecting data on the location of the optical data receiving mechanism either through an imaging technology such as ultrasound or another modality or through a remote positioning mechanism similar to a global positioning system. Data on the location of the optical data receiving mechanism can be transmitted to a computational device as shown in block 314.

Data on the location of the optical data receiving mechanism 132 received or acquired by the imager/localizer 140 is transmitted or delivered to the computational device 104 through the imager/localizer 126 and to the system bus 105. From the system bus 105, the data from the imager/localizer can be transmitted or coupled to other components of the computational device 104, including the human/machine interface 108, the processor 106 and the memory 112.

Similar to the data collected by the optical data receiving mechanism as described above, imager/localizer data is typically stored in the data component 114 of the memory 112 and is preprocessed and analyzed by the data preprocessing/image analysis software 118. It should be recognized, however, that the imager/localizer can include its own data preprocessing imager analysis software, as well as system software, such that data delivered to computational device 104 from the imager/localizer may already be preprocessed.

In one aspect, preprocessed data both from the optical data receiving mechanism and from the imager/localizer are used by the estimation/reconstruction software 120 to compute parameters/features/distributions of the light source in the subject as well as tissue parameters of the subject as shown in block 318. Exemplary algorithms that can be used to perform the estimation/reconstruction are described above. The above discussed formulas can be used in combination with an interactive minimization or fitting technique.

### EXAMPLES

The following example is put forth so as to provide those of ordinary skill in the art with a complete disclosure and description of how the systems, methods, and devices claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, temperature is in ° C., and pressure is at or near atmospheric.

### EXAMPLE 1

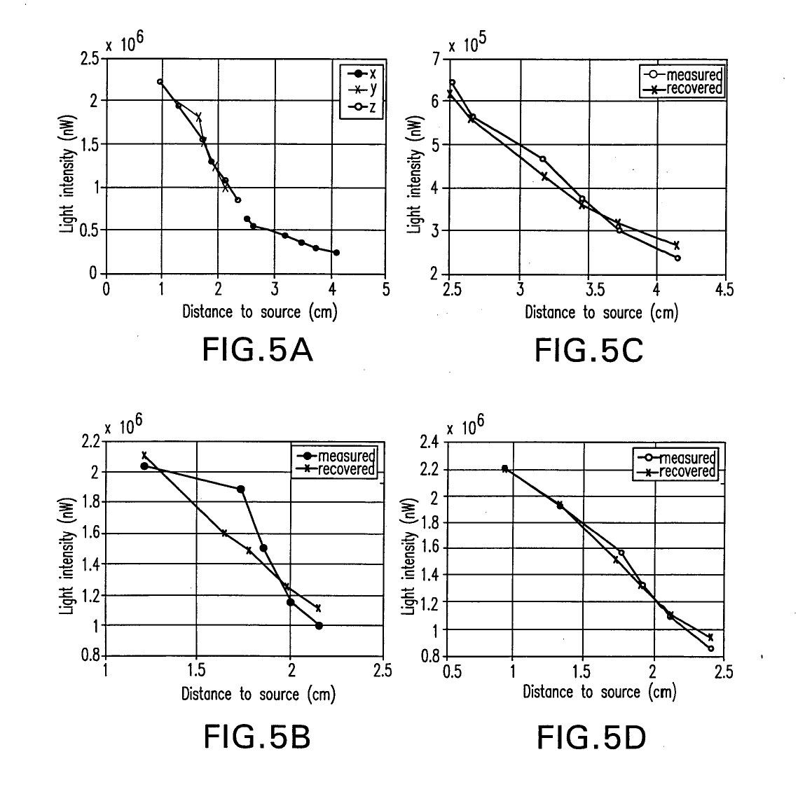

The forward process, measurement and reconstruction procedures were numerically and physically simulated. An optical fiber biopsy needle of bare fiber tip with flat end (Polymicro Technologies Inc., Phoenix, Ariz.) was used. A 6.5×6×6 cm3 phantom was made of 9 g agar in 550 ml distilled water. One red firefly light stick was used with one tip of length 0.5 cm being exposed to emit light and the other part covered with black tape. Three orthogonal insertions were performed under monitoring with a digital camera (Nikon DIX) with a resolution 0.4 mm positioned at a distance 500 mm. A ruler was in the field of view for localizing the measurement point. The data are shown in FIG. 5(a). The recovered optical and geometrical parameters were used in the forward model to simulate the measured data by (32) so that the true source center and total energy can be estimated. FIGS. 5(b-d) indicate a good agreement between the measured and recovered data.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A system for reconstructing a light source distribution within a subject, comprising:
an optical data receiving mechanism positionable at least at one location within the subject wherein the optical data receiving mechanism is configured to acquire signal data from a light source located within a subject; and
a computational device configured to receive signal data acquired by the optical data receiving mechanism and to reconstruct the light source distribution from at least a portion of the received data.

2. The system of claim 1, wherein the optical data receiving mechanism is configured to acquire signal data from a bioluminescent light source located within the subject.

3. The system of claim 1, wherein the optical data receiving mechanism is configured to acquire signal data from a florescent light source located within the subject.

4. The system of claim 1, wherein the optical data receiving mechanism comprises at least one detection fiber for acquiring signal data.

5. The system of claim 4, wherein the optical data receiving mechanism further comprises at least one source fiber for transmitting light energy from the optical data receiving mechanism into the subject.

6. The system of claim 5, wherein the detection fiber and the source fiber are positioned within a housing structure, wherein the housing structure is configured for advancement through tissue of the subject.

7. The system of claim 6, wherein the housing structure comprises an elongated tubular structure having a first end, wherein the first end is configured for penetrating tissue of the subject.

8. The system of claim 7, wherein a portion of the first end is further configured to allow transmission of light energy therethrough.

9. The system of claim 7, wherein the source fiber and the detection fiber are positioned within the lumen of the tubular structure.

10. The system of claim 1, further comprising a localizing device for detecting the position of the optical data receiving mechanism when the optical data receiving mechanism is located within the subject, wherein the localizing device can be operatively connected to the computational device for transmitting the positional data thereto.

11. The system of claim 10, wherein the localizing device is selected from the group consisting of an ultrasound imaging modality, a computed tomography imaging modality, a magnetic resonance imaging modality, and a remote positioning detection mechanism.

12. The system of claim 11, wherein the localizing device is a remote positioning mechanism and wherein the optical data receiving mechanism further comprises a signal generator for producing a signal detectable by the remote positioning system.

13. The system of claim 10, wherein the localizing device is further configured to provide anatomical data from the subject and is configured to transmit the anatomical data to the computational device.

14. The system of claim 13, wherein the localizing device is selected from the group consisting of an ultrasound imaging modality, a computed tomography imaging modality, and a magnetic resonance imaging modality.

15. The system of claim 14, wherein the anatomical data comprises data acquired using the ultrasound imaging modality, the computed tomography imaging modality, or the magnetic resonance imaging modality.

16. The system of claim 5, wherein the source fiber is operatively connected to a laser light source.

17. The system of claim 16, further comprising a fiber bifurcation apparatus for splitting the laser light energy into a source path and into a reference path.

18. The system of claim 17, wherein in the source path is operatively connected to the source fiber of the optical data receiving mechanism.

19. The system of claim 18, further comprising a transmitter for transmitting the light energy in the reference path to the computational device.

20. The system of claim 19, wherein the transmitter comprises at least one optical fiber.

21. The system of claim 1, wherein the computational device is operatively connected to the optical data receiving device by a transmitter comprising at least one optical fiber.

22. The system of claim 1, wherein the transmitter further comprises signal detector device.

23. The system of claim 22, wherein the signal detector device is a charged-coupled device (CCD) camera or a photon detecting device.

24. The system of claim 1, wherein the computational device further comprises computer readable code for reconstructing the light source distribution utilizing an inverse source approach.

25. The system of claim 10, wherein the computational device further comprises computer readable code for reconstructing the light source distribution utilizing an inverse source approach.

26. The system of claims 24 or 25, wherein the computer readable code performs the steps of solving a forward model of light flux from one or more light source.

27. The system of claim 26, wherein the forward model of light flux is provided by a radiative transport algorithm or a diffusion approximation algorithm.

28. The system of claim 13, wherein the computational device further comprises computer readable code for reconstructing the light source distribution utilizing an inverse source approach.

29. An system for estimating a light source feature within a subject, comprising:
an optical data receiving mechanism positionable at least at one location within the subject and configured to acquire signal data from a light source located within a subject; and
a computational device configured to receive signal data acquired by the optical data receiving mechanism and to estimate the light source feature from at least a portion of the received data.

30. The system of claim 29, wherein the estimated light source feature is selected from the group consisting of the center of the light source, the total energy of the light source, the absorption properties of the tissue around the light source, and the scattering properties of the tissue around the light source.

31. The system of claim 29, wherein the computational device further comprises computer readable code for estimating the light source feature utilizing an inverse source approach.

32. The system of claim 31, wherein the computer readable code performs the steps of solving a forward model of light flux from one or more light source.

33. A method of reconstructing a light source distribution within a subject, comprising:
positioning an optical data receiving mechanism within the subject, wherein the optical data receiving mechanism acquires signal data from a light source;
transmitting at least a portion of the acquired signal data from the optical data receiving mechanism to a computational device; and
reconstructing the distribution of the light source using at least a portion of the transmitted data.

34. The method of claim 33, wherein the step of reconstructing comprises performing an inverse source approach on at least a portion of the transmitted data using a computational device.

35. The method of claim 34, wherein the computational device performs the steps of solving a forward model of light flux from one or more light source.

36. The method of claim 34, wherein the forward model of light flux is provided by a radiative transport algorithm or a diffusion approximation algorithm.

37. The method of claim 33, further comprising:
acquiring anatomical data from the subject and positional data from the optical data receiving mechanism located within the subject;
transmitting at least a portion of the acquired signal data, anatomical data, and positional data to a computational device; and
processing at least a portion of the transmitted signal data, anatomical data, and positional data to reconstruct the distribution of the light source.

38. The method of claim 37, wherein the step of reconstructing comprises performing an inverse source approach on at least a portion of the transmitted data using a computational device.

39. The method of claim 38, wherein the computational device performs the steps of solving a forward model of light flux from one or more light source.

40. The method of claim 39, wherein the forward model of light flux is provided by a radiative transport algorithm or a diffusion approximation algorithm.

41. A method for estimating a light source feature within a subject, comprising:
positioning an optical data receiving mechanism within the subject, wherein the optical data receiving mechanism acquires signal data from a light source;
transmitting at least a portion of the acquired signal data from the optical data receiving mechanism to a computational device; and
estimating a feature of the light source using at least a portion of the transmitted data by estimating the distribution of the light source.

42. The method of claim 41, wherein the step of reconstructing comprises performing an inverse source approach on at least a portion of the transmitted data using a computational device.

43. The method of claim 42, wherein the computational device performs the steps of solving a forward model of light flux from one or more light source.

44. The method of claim 43, wherein the forward model of light flux is provided by a radiative transport algorithm or a diffusion approximation algorithm.

45. The method of claim 41, further comprising:
acquiring anatomical data from the subject and positional data from the optical data receiving mechanism located within the subject;
transmitting at least a portion of the acquired signal data, anatomical data, and positional data to a computational device; and
processing at least a portion of the transmitted signal data, anatomical data, and positional data to determine the distribution of the light source.
